(19) United States
(12) Patent Application Publication
(10) Pub. No.: US 2023/0059908 A1
(43) Pub. Date: Feb. 23, 2023

(54) VENTILATION MONITORING SYSTEMS AND METHODS

(71) Applicant: COVIDIEN LP, Mansfield, MA (US)

(72) Inventor: Julio Jenaro, Roscam (IE)

(21) Appl. No.: 17/888,034

(22) Filed: Aug. 15, 2022

Related U.S. Application Data
(60) Provisional application No. 63/235,447, filed on Aug. 20, 2021.

(51) Int. Cl. A61M 16/00; G16H 10/60

(52) U.S. Cl. CPC A61M 16/026; A61M 16/0051; G16H 10/60; A61M 2230/43

(57) ABSTRACT

A ventilation monitoring system includes one or more processors configured to receive data from a sensor positioned along a breathing circuit during a mechanical ventilation procedure for a patient. The one or more processors are also configured to analyze the data to determine respective concentrations of ions at the sensor and to calculate a detected ratio of the respective concentrations of the ions at the sensor. The one or more processors are also configured to compare the detected ratio to an expected ratio to determine a condition of the patient and to output a notification to indicate the condition of the patient.

### CROSS-REFERENCE TO RELATED APPLICATION

The present application claims the benefit of and priority to U.S. Provisional Patent Application Ser. No. 63/235,447, filed on Aug. 20, 2021, the entire content of which is incorporated herein by reference.

### BACKGROUND

The present disclosure relates generally to medical devices and, more particularly, to systems and methods for monitoring a condition of a patient during a mechanical ventilation procedure.

This section is intended to introduce the reader to various aspects of art that may be related to the present disclosure, as described and/or claimed below. This discussion is believed to be helpful in providing the reader with background information to facilitate a better understanding of the various aspects of the present disclosure. Accordingly, it should be understood that these statements are to be read in this light, and not as admissions of prior art.

During a mechanical ventilation procedure, a tube or other medical device may be used to control a flow of gases, food, fluids, or other substances into a patient. For example, a tracheal tube may be used to control the flow of gases through a trachea of the patient and into the lungs of the patient. In many instances, when the tracheal tube is inserted into a body passage of the patient, it is desirable to provide a seal between the outside of the tracheal tube and an interior of the body passage of the patient. In this way, substances can only flow through the body passage of the patient via the tracheal tube, which may enable a medical practitioner to maintain control over a type and an amount of gases flowing into and out of the patient.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

In one embodiment, a ventilation monitoring system includes one or more processors configured to receive data from a sensor positioned along a breathing circuit during a mechanical ventilation procedure for a patient. The one or more processors are also configured to analyze the data to determine respective concentrations of ions at the sensor and to calculate a detected ratio of the respective concentrations of the ions at the sensor. The one or more processors are also configured to compare the detected ratio to an expected ratio to determine a condition of the patient and to output a notification to indicate the condition of the patient.

In an embodiment, a method of operating a ventilation monitoring system includes receiving, at one or more processors, data from a sensor positioned along a breathing circuit during a mechanical ventilation procedure for a patient. The method also includes analyzing, with the one or more processors, the data to determine a detected ratio of respective concentrations of ions at the sensor. The method also includes comparing, with the one or more processors, the detected ratio to an expected ratio. The method also includes outputting a notification in response to determining that the detected ratio does not correspond to the expected ratio.

In an embodiment, a ventilation monitoring system includes a tracheal tube having a sensor configured to generate data indicative of respective concentrations of ions. The ventilation monitoring system also includes a ventilator configured to couple to the tracheal tube and having one or more processors configured to instruct delivery of respiratory gases through the tracheal tube during a mechanical ventilation procedure for a patient. The one or more processors are also configured to receive the data from the sensor during the mechanical ventilation procedure, analyze the data to calculate a detected ratio of the respective concentrations of the ions, compare the detected ratio to an expected ratio to determine a condition of the patient, output a notification via a display screen of the ventilator to indicate the condition of the patient.

### BRIEF DESCRIPTION OF THE DRAWINGS

Advantages of the disclosed techniques may become apparent upon reading the following detailed description and upon reference to the drawings in which:

FIG. 1 is a schematic illustration of a ventilation monitoring system that is configured to monitor a condition of a patient during a mechanical ventilation procedure, in accordance with certain embodiments of the disclosure;

FIG. 2 is a flow diagram of a method of operating the ventilation monitoring system of FIG. 1, in accordance with certain embodiments of the disclosure;

FIG. 3 is a schematic illustration of a graphical user interface that may be presented via a display screen of the ventilation monitoring system of FIG. 1, in accordance with certain embodiments of the disclosure;

FIG. 4 is a perspective view of a tracheal tube that may be used as part of the ventilation monitoring system of FIG. 1, in accordance with certain embodiments of the disclosure; and

FIG. 5 is a perspective view of a mask that may be used as part of the ventilation monitoring system of FIG. 1, in accordance with certain embodiments of the disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

A tracheal tube is configured to be inserted into a trachea of a patient and is used to transfer gases from a ventilator to the patient. After positioning the tracheal tube at a desired position within the trachea, a cuff is inflated around the tracheal tube to seal a tracheal passage at a pressure that sufficiently seals an airway of the patient. In some embodiments, the tracheal tube may have two cuffs, which may facilitate use of the tracheal tube to transfer gases from the ventilator to one lung of the patient. In such cases, one cuff may seal the tracheal passage and another cuff may seal a bronchial passage of the patient. The tracheal tube with the two cuffs may also be referred to as an endobronchial tube. In some cases, a mask (e.g., non-invasive face mask) may be use to transfer gases from the ventilator to the patient.

It is presently recognized that active transport of ions, such as active transport of sodium (Na⁺) and potassium (K⁺), across an alveolar epithelium of the patient is an important factor in achieving a positive outcome for the patient undergoing mechanical ventilation. For example, the active transport of sodium and potassium is important for clearance of fluid from the lungs of the patient, as well as reduced incidence of alveolar lung disease (ALD), acute lung injury (ALI), acute respiratory distress syndrome (ARDS), various types of pulmonary edema, and/or various other conditions.

Accordingly, the embodiments disclosed herein relate generally to a ventilation monitoring system that includes a sensor that is configured to detect a concentration of one or more ions (e.g., sodium and potassium ions). The sensor may be mounted or integrated into the tracheal tube or another device (e.g., the mask) that is exposed to an airway of the patient (e.g., in a breathing circuit that transfers gases from the ventilator to the patient). For example, the sensor may be mounted or integrated into a tip portion of the tracheal tube, such that the sensor is placed in the bronchus of the patient during the mechanical ventilation procedure. The sensor may be communicatively coupled to processing circuitry that is configured to analyze data from the sensor to evaluate a condition of the patient and/or to provide a notification (e.g., audible alarm or text message) to indicate the condition of the patient. Advantageously, the ventilation monitoring system may analyze the data and provide the notification in real-time (e.g., real-time or substantially real-time) during the mechanical ventilation procedure. Thus, the medical professional may have more information during the mechanical ventilation procedure, which may enable the medical professional to adjust treatment of the patient in a way that is beneficial to the patient.

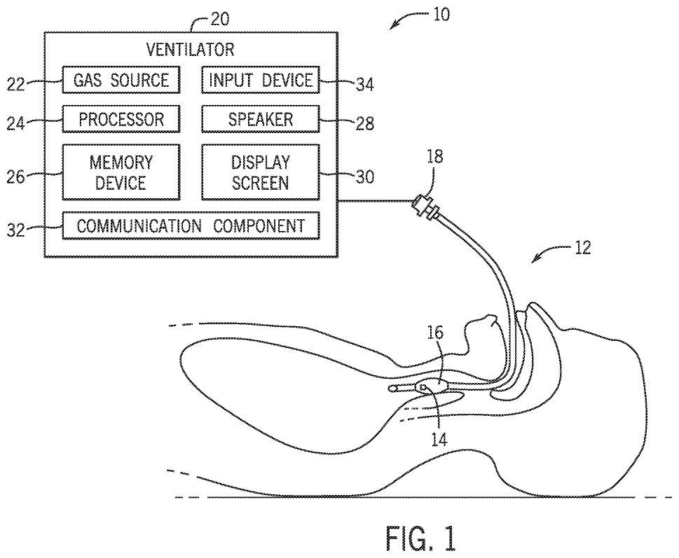

FIG. 1 is a schematic illustration of a ventilation monitoring system 10 (also referred to herein “the system”) that is configured to monitor a patient during a mechanical ventilation procedure, in accordance with certain embodiments of the disclosure. As shown, the system 10 includes a tracheal tube 12 (e.g., tracheal tube assembly) that is configured to be inserted into a body passage of a patient, and a sensor 14 (e.g., analyte sensor) that is associated with (e.g., mounted on or integrated into) the tracheal tube 12. The tracheal tube 12 also includes at least one cuff 16 that is configured to inflate to form a seal between a tube portion (e.g., flexible tube portion) of the tracheal tube 12 and the body passage of the patient. The tracheal tube 12 further includes a connector 18 that is configured to connect to a ventilator 20, which is configured to provide respiratory gases from a gas source 22 into the tracheal tube 12.

The system 10 may include processing circuitry that is configured to receive and to process data from the sensor 14. The processing circuitry may include one or more processors 24 that are configured to process data, such as data generated by the sensor 14. More particularly, the one or more processors 24 may be used to execute processor-executable instructions (e.g., firmware or software) stored on a memory device 26 to process data generated by the sensor 14 to determine a condition of the patient (e.g., of the alveolar epithelium of the patient), generate a notification that is output via a speaker 28 and/or a display screen 30, and/or to carry out any of a variety of techniques in accordance with the present disclosure.

The one or more processors 24 may include multiple microprocessors, one or more “general-purpose” microprocessors, one or more special-purpose microprocessors, and/or one or more application specific integrated circuits (ASICS), or some combination thereof. For example, the one or more processors 24 may include one or more reduced instruction set (RISC) processors. The memory device 26 may include a volatile memory, such as random access memory (RAM), and/or a nonvolatile memory, such as read-only memory (ROM). It should be appreciated that the memory device 26 may include flash memory, a hard drive, or any other suitable optical, magnetic, or solid-state storage medium, other hardware memory, or a combination thereof. The memory device 26 may store a variety of information (e.g., data, instructions) and may be used for various purposes.

The processing circuitry may also be coupled to a communication component 32 that is configured to facilitate communication with other monitors or systems, such as hospital monitors or systems. The processing circuitry may further include or be coupled to one or more input devices 34 (e.g., touch input devices) that are configured to receive inputs from the medical professional, such as inputs to clear the notification, to set threshold(s), input patient information, or the like.

In the illustrated embodiment, the processing circuitry is located in the ventilator 20, and thus, the processing circuitry may also be configured to control ventilation parameters related to delivery of the respiratory gases from the gas source 22. Furthermore, in such cases, the speaker 28 and/or the display screen 30 may be configured to provide the notification related to the condition of the patient along with other information related to the delivery of the respiratory gases from the gas source 22 (e.g., the ventilation parameters).

However, it should be appreciated that the processing circuitry may be located in a separate monitor (e.g., separate computing system) that is distinct from the ventilator 20. For example, the separate monitor may be configured to analyze the data from the sensor 14, to control inflation of the at least one cuff 16, and/or to manage other aspects of the mechanical ventilation procedure. As another example, the separate monitor may be a specialized, stand-alone monitor that is configured only to analyze the data from the sensor 14 and perform related functions (e.g., provide the notification). In some embodiments that include the separate monitor in addition to the ventilator 20, the separate monitor may include its own communication component to communicate with the sensor 14 and/or the ventilator 20 (and/or other monitors or systems) via a wired or wireless connection and/or its own input devices to receive inputs. Furthermore, the separate monitor may include its own speaker and/or display screen to provide the notification.

Regardless of the location of the processing circuitry, the sensor 14 is configured to provide the data to the processing circuitry via a wired or wireless connection. For example, the sensor 14 may provide the data via an electrical cable that extends from the sensor 14 to the processing circuitry, and the electrical cable may be embedded (e.g., encapsulated) in material that forms the tube portion of the tracheal tube 12. As another example, the sensor 14 may provide the data via a wireless protocol, such as Bluetooth or WiFi.

Additionally, the sensor 14 may be powered via any of a variety of power sources. For example, the sensor 14 may be powered via a wired connection formed by a power cable that extends from a power source to the sensor 14, and the power cable may be embedded in the material that forms the tube portion of the tracheal tube 12. As another example, the sensor 14 may be powered by a battery that is positioned proximate to the sensor 14, such as mounted on or integrated into the tracheal tube 12. In some embodiments, the sensor 14 may be powered by a wireless connection, such as via power harvesting circuitry that is mounted on or integrated into the tracheal tube 12. The power harvesting circuitry may be configured to harvest power via radiofrequency signals, such as via near field communication (NFC) signals. It should be appreciated that variations of these components and configurations are envisioned. For example, the battery or the power harvesting circuitry may be mounted at a proximal end portion of the tracheal tube 12 (e.g., near the connector 18), and the battery or the power harvesting circuitry may be connected to the sensor 14 that is positioned at a distal end portion of the tracheal tube 12 (e.g., near the tip) via the power cables embedded in the material that forms the tube portion of the tracheal tube 12.

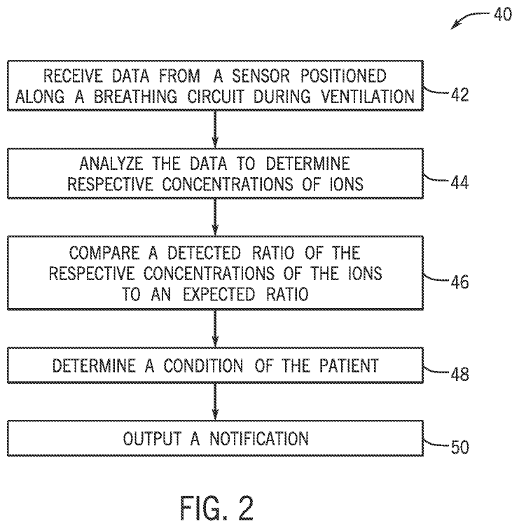

FIG. 2 is a flow diagram of a method 40 of operating the system 10 of FIG. 1, in accordance with certain embodiments of the disclosure. The method 40 disclosed herein includes various steps represented by blocks. It should be noted that at least some steps of the method 40 may be performed as an automated procedure by a system, such as the system 10. Although the flow diagram illustrates the steps in a certain sequence, it should be understood that the steps may be performed in any suitable order and certain steps may be carried out simultaneously, where appropriate. Additionally, steps may be added to or omitted from of the method 40. Further, certain steps or portions of the method 40 may be performed by separate devices. For example, a first portion of a method 40 may be performed by a processor of a separate monitor, while a second portion of the method may be performed by the processor 24 of the ventilator 20. In addition, insofar as steps of the method 40 disclosed herein are applied to received signals or data, it should be understood that the received signals or data may be raw signals or data or processed signals or data. That is, the method 40 may be applied to an output of the received signals or data.

Generally, the method 40 includes steps that use the sensor 14 to detect a concentration of one or more ions (e.g., respective concentrations of sodium and potassium ions) along the breathing circuit during a mechanical ventilation procedure. The processing circuitry may receive and analyze data from the sensor 14 to determine a condition of the patient and to provide a notification to indicate the condition of the patient.

More particularly, in step 42, the processing circuitry may receive data from the sensor 14 that is positioned along the breathing circuit during the mechanical ventilation procedure. The sensor 14 may be coupled to the tracheal tube 12 that is inserted into the body passage of the patient or positioned at any other suitable location between the lungs of the patient and the ventilator 20. The data from the sensor 14 may indicate a concentration of one or more ions, specifically respective concentrations of sodium and potassium ions, at the location of the sensor 14. For example, body fluids (e.g., mucus, saliva) may accumulate on the sensor 14, and the sensor 14 may detect the respective concentrations of the sodium and potassium ions in the body fluids. Then, the sensor 14 may provide data indicative of the respective concentrations of the sodium and potassium ions in the body fluids to the processing circuitry.

In step 44, the processing circuitry may analyze the data from the sensor 14. In healthy lungs (e.g., with adequate clearance of fluid from the lungs; without alveolar lung disease (ALD), acute lung injury (ALI), acute respiratory distress syndrome (ARDS), various types of pulmonary edema, and/or various other conditions), the sodium and potassium ions are expected to be present in respective concentrations that provide a certain ratio (e.g., expected ratio or range of expected ratios). For example, the sodium and potassium ions may be expected to be present with respective concentrations of 2 to 4 sodium ions for every 1 to 3 potassium ions, 2 or 3 sodium ions for every 1 to 2 potassium ions, 3 to 4 sodium ions for every 2 to 3 potassium ions, or 3 sodium ions for every 2 potassium ions.

Some patients may have lung conditions prior to beginning the mechanical ventilation procedure or may experience changes to their lungs during the mechanical ventilation procedure. For these patients, the ratio (e.g., the detected or the current ratio) of the respective concentrations of the sodium and potassium ions may be different than the expected ratio that is associated with healthy lungs. Thus, the processing circuitry may analyze the data from the sensor 14 to calculate the detected ratio of the respective concentrations of the sodium and potassium ions.

In step 46, the processing circuitry may compare the detected ratio to the expected ratio, which the processing circuitry may access (e.g., from a memory device). As noted above, the sodium and potassium ions are expected to be present in respective concentrations that provide a certain ratio or balance. Accordingly, the expected ratio may be set as respective concentrations of 2 to 4 sodium ions for every 1 to 3 potassium ions, 2 or 3 sodium ions for every 1 to 2 potassium ions, 3 to 4 sodium ions for every 2 to 3 potassium ions, or 3 sodium ions for every 2 potassium ions. However, it should be appreciated that the expected ratios provided herein are merely non-limiting examples and that the expected ratio may be set to be any suitable ratio that relates the respective concentrations of sodium ions to potassium ions (or potassium ions to sodium ions).

In step 48, the processing circuitry may determine the condition of the patient based on the comparison between the detected ratio and the expected ratio. For example, if the detected ratio corresponds to the expected ratio (e.g., matches the expected ratio or is within the range of expected ratios), then the processing circuitry may determine that the condition is healthy. However, if the detected ratio does not correspond to the expected ratio (e.g., does not match the expected ratio or is outside of the range of expected ratios), then the processing circuitry may determine that the condition is unhealthy or that the condition warrants attention.

In step 50, the processing circuitry may output a notification based on the condition of the patient. The notification may include an audible alarm (e.g., via the speaker 28) and/or a text message (e.g., via the display screen 30) that conveys information about the condition of the alveolar epithelium of the patient. The notification may vary based on a difference between the detected ratio and the expected ratio. The difference may indicate a severity of the condition of the patient, and thus, the notification may convey the severity of the condition of the patient. For example, the notification may be a first audible alarm (e.g., lower volume or rate) for a first, lower difference, and the notification may be a second audible alarm (e.g., higher volume or rate) for a second, higher difference. Similarly, the notification may include a first visual alert (e.g., first color, such as yellow; first symbol) for the first, lower difference, and the notification may include a second visual alert (e.g., second color, such as red; second symbol) for the second, higher difference. Generally, the notification may include different sounds, text, colors, and/or symbols based on the severity of the condition of the patient. Various examples of the notification, and the information in the notification, are discussed in more detail herein.

It should be appreciated that the method 40 may include additional steps and/or features to facilitate effective monitoring techniques. For example, the method 40 may include steps that provide for customized or patient-specific monitoring. To achieve this, the method 40 may include receiving patient data at the processing circuitry prior to or during the mechanical ventilation procedure. The patient data may be input by the medical professional, such as via the one or more input devices 34, and/or the patient data may be accessed from one or more other systems or devices, such as via the communication component 32. In such cases, the patient data may include an age of the patient, a gender of the patient, physiological parameters of the patient (e.g., heart rate, oxygen saturation, prior ratio(s) of sodium and potassium ions during prior mechanical ventilation(s)), a smoking history of the patient, an illness of the patient that led to treatment with the mechanical ventilation procedure, and/or any other relevant factors. In some embodiments, the patient data may be used to calculate the expected ratio for the patient for the mechanical ventilation procedure. For example, the processing circuitry may utilize one or more algorithms that receive the patient data as inputs to calculate the expected ratio for the patient. In such cases, a standard expected ratio (e.g., theoretical or experimental ratio based on modeled or empirical data; 2 to 4 sodium ions for every 1 to 3 potassium ions) may be applied during mechanical ventilation procedures in the absence of the patient data, and the standard expected ratio may be increased or decreased based on the patient data during mechanical ventilation procedures after receipt of the patient data.

In some embodiments, the method 40 may include establishing a baseline ratio between the respective concentrations of the sodium and potassium ions. The baseline ratio for the patient may be considered to be another type of patient data that facilitates customized or patient-specific monitoring. The baseline ratio for the patient may be established using respective concentrations of the sodium and potassium ions taken during an initial time point and/or at multiple time points over an initial period of the mechanical ventilation procedure (e.g., the first 1, 2, 3, 4, 5, 10, 15, or 30 minutes). For example, the baseline ratio may be calculated based on an average or a median of multiple detected ratios over the initial time period. Then, the baseline ratio may be set to be the expected ratio for the patient for the remainder of the mechanical ventilation procedure.

In some embodiments, the baseline ratio may be used to calculate the expected ratio for the patient for the remainder of the mechanical ventilation procedure. For example, the processing circuitry may utilize one or more algorithms that calculate the expected ratio for the patient to be a range about the baseline ratio and/or a range that encompasses some or all of the multiple detected ratios over the initial time period. As another example, the processing circuitry may utilize one or more algorithms that adjust the standard expected ratio (e.g., increase or decrease) based on the baseline ratio for the patient. It should be appreciated that the processing circuitry may employ one or more algorithms that utilize both the baseline ratio and other types of patient data together to calculate the expected ratio for the patient.

In some embodiments, the method 40 may include steps that determine trends in the detected ratio for the patient over time and that determine the condition of the patient based on the trends. The method 40 may include steps that provide predictions with respect to a future condition of the patient (e.g., at a future time). In particular, the processing circuitry may monitor the trends in the detected ratio for the patient over time during the mechanical ventilation procedure. Then, the processing may use machine learning (e.g., deep learning) to predict the future condition of the patient at the future time during the mechanical ventilation procedures. These techniques may utilize other inputs, such as the baseline ratio, the other types of patient data, and/or the ventilation parameters to predict the future condition of the patient. As used herein, machine learning refers to algorithms and statistical models that may be used to perform a specific task without using explicit instructions, relying instead on patterns and inference. In particular, machine learning generates a mathematical model based on data (e.g., sample or training data, historical data) in order to make predictions without being explicitly programmed to perform the task.

In some embodiments, the method 40 may include steps that provide a recommendation (e.g., a treatment recommendation) for the patient based on the condition and/or the future condition of the alveolar epithelium. For example, the recommendation may include an adjustment to the ventilation parameters, treatment with serum potassium or other chemical compound, and/or close monitoring for some period of time. The recommendation may be general (e.g., adjust ventilation parameters or treat with chemical compound) or specific (e.g., adjust to certain ventilation parameters or treat with certain type and/or amount of chemical compound). The recommendation may be output as part of the notification, such as via the text message on the display screen 30.

Furthermore, the method 40 may includes steps that monitor an effectiveness of the adjustment to the ventilation parameters and/or the treatment. For example, the processing circuitry may receive an input that indicates completion of the adjustment to the ventilation parameters and/or the treatment. Then, the processing circuitry may monitor the detected ratio and may determine whether the detected ratio behaves in an expected manner following the adjustment to the ventilation parameters and/or the treatment. For example, it may be expected that the detected ratio stops increasing and remains stable for a period of time following the treatment, and the processing circuitry may monitor the detected ratio and may determine that the patient is responding to the treatment if the detected ratio stops increasing and remains stable for the period of time following the treatment. However, the processing circuitry may determine that the patient is not responding to the treatment if the detected ratio continues to increase and/or does not remain stable for an entirety of the period of time following the treatment. The processing circuitry may provide an output to indicate whether the response to the adjustment to the ventilation parameters and/or the treatment is expected, and the output may be included as part of the notification, such as via the text message on the display screen 30. It should also be appreciated that the processing circuitry may output the expected response to the adjustment to the ventilation parameters and/or the treatment (e.g., alongside the recommendation) to assist the medical professional.

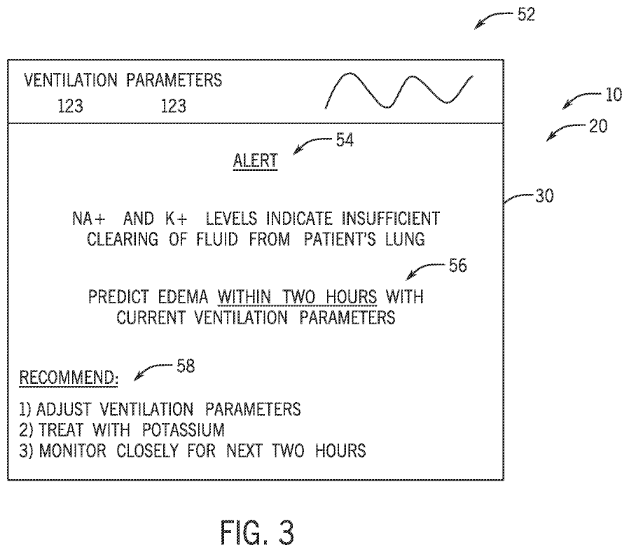

FIG. 3 is a schematic illustration of a graphical user interface (GUI) 52 that may be presented via a display screen, such as via the display screen 30 of the system 10 of FIG. 1, in accordance with certain embodiments of the disclosure. As shown, and particularly when the GUI 52 is presented via the display screen 30 that is part of the ventilator 20, the GUI 52 may include the ventilation parameters (e.g., mode, respiratory rate, flow rate, tidal volume, waveform). The GUI 52 may also include the notification, which may be presented as an alert 54 in response to the detected ratio failing to correspond to the expected ratio.

In some embodiments, the GUI 52 may include a prediction 56 related to the future condition of the alveolar epithelium. The prediction 56 may be presented in any of a variety of ways, such as with an indication of the future condition and/or the future time at which the future condition is expected to occur. In some embodiments, the GUI 52 may also include a recommendation 58, which may include an adjustment to the ventilation parameters, treatment with a chemical compound, and/or close monitoring for some period of time.

It should be appreciated that the GUI 52 in FIG. 3 is intended to be only one non-limiting example. In operation, the GUI 52 may omit some of the information that is shown in FIG. 3 and/or include additional information that is not shown in FIG. 3. Furthermore, while the alert 54 is shown to include a text message that specifically refers to ion levels, the alert 54 may include a text message that conveys other details and/or may have any suitable format. While the prediction 56 refers to the future condition of edema and the future time is shown to be two hours, it should be appreciated that the prediction 56 may include any of a variety of future conditions and/or future times (e.g., one, two, three, four, five, ten, 24 hours or more). Additionally, it should be appreciated that the recommendation 58 may include any suitable action and may be presented in any suitable format.

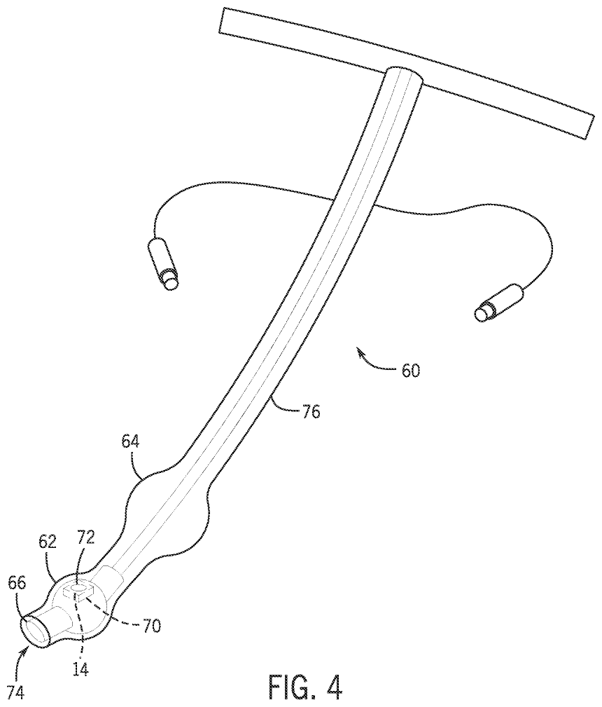

FIG. 4 is a perspective view of a tracheal tube 60 that may be used as part of the system 10 of FIG. 1, in accordance with certain embodiments of the disclosure. In FIG. 4, the tracheal tube 60 may have any of the features of the tracheal tube 12 shown in FIG. 1 and described herein. However, that the tracheal tube 60 has a first cuff 62 (e.g., distal cuff) and a second cuff 64 (e.g., proximal cuff) to facilitate insertion of a tip 66 of the tracheal tube 60 into a bronchus of the patient. The first cuff 62 and the second cuff 64 allow the tracheal tube 60 to isolate one lung of the patient and to deliver the respiratory gases from the ventilator to the one lung of the patient.

As shown, the sensor 14 is exposed at an exterior surface of the first cuff 62. More particularly, a body 70 of the sensor 14 may be embedded in a material (e.g., flexible material) that forms the first cuff 62, and a detection pad 72 of the sensor 14 may be exposed at the exterior surface of the first cuff 62. This configuration may be achieved by molding the sensor 14 into the first cuff 62 (e.g., during manufacturing) and then removing a portion of the material of the first cuff 62 that covers the detection pad 72 of the sensor 14. However, it should be appreciated that this configuration may be achieved via other manufacturing techniques and/or the sensor 14 may be joined to the tracheal tube 60 in other configurations. It should also be appreciated that the sensor 14 may be positioned at any of a variety of other locations along the tracheal tube 60, such in an interior passage 74 of the tracheal tube 60, along an exterior surface or in the interior passage 74 at a tube portion 76 of the tracheal tube 60 (e.g., between the first cuff 62 and the tip 66 of the tracheal tube 60; between the first cuff 62 and the second cuff 64; between the second cuff 64 and a proximal end of the tracheal tube 60), or the like. In any case, with the detection pad 72 of the sensor 14 exposed to the body fluids of the patient, the detection pad 72 of the sensor 14 may detect the respective concentrations of the sodium and potassium ions. It should be appreciated that the sensor 14 may be mounted to the tracheal tube 12 shown in FIG. 1 in a similar manner.

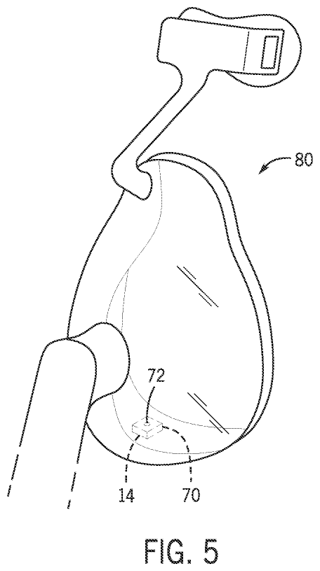

FIG. 5 is a perspective view of a mask 80 (e.g., face mask) that may be used as part of the system 10 of FIG. 1, in accordance with certain embodiments of the disclosure. The mask 80 may enable the medical professional to utilize a non-invasive mechanical ventilation procedure for the patient. Advantageously, the mask 80 may include the sensor 14 exposed at an interior surface of the mask 80. In some embodiments, the body 70 of the sensor 14 may be embedded in a material (e.g., rigid or flexible material) that forms the mask 80, and the detection pad 72 of the sensor 14 may be exposed at the interior surface of the mask 80. This configuration may be achieved by molding the sensor 14 into the mask 80 (e.g., during manufacturing) and then removing a portion of the material of the mask 80 that covers the detection pad 72 of the sensor 14. However, it should be appreciated that this configuration may be achieved via other manufacturing techniques and/or the sensor 14 may be joined to the mask 80 in other configurations. It should also be appreciated that the sensor 14 may be positioned at any of a variety of other locations about the mask 80, such in an interior passage of a connector 82 of the mask 80. In some embodiments, it may be desirable to position the sensor 14 along a bottom portion (e.g., bottom-most portion or surface) of the interior surface of the mask 80, as then the body fluids may pool or accumulate over the detection pad 72. In any case, with the detection pad 72 of the sensor 14 exposed to the body fluids of the patient, the detection pad 72 of the sensor 14 may detect the respective concentrations of the sodium and potassium ions.

The embodiments disclosed herein relate generally to a ventilation monitoring system that includes a sensor that is configured to detect respective concentrations of ions (e.g., sodium and potassium ions) during a mechanical ventilation procedure. Advantageously, the ventilation monitoring system may assess the respective concentrations to determine whether the ions are in an appropriate or expected balance, which indicates whether the alveolar epithelium of the patient is functioning properly. Thus, the respective concentrations of the ions may also indicate a condition of the patient or enable prediction of a future condition of the patient. The ventilation monitoring system may also provide notifications, such as notifications that indicate the condition of the patient in real-time or the future condition of the patient, during the mechanical ventilation procedure.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

What is claimed is:

1. A ventilation monitoring system, comprising:
one or more processors configured to:
receive data from a sensor positioned along a breathing circuit during a mechanical ventilation procedure for a patient;
analyze the data to determine respective concentrations of ions at the sensor and to calculate a detected ratio of the respective concentrations of the ions at the sensor;
compare the detected ratio to an expected ratio to determine a condition of the patient; and
output a notification to indicate the condition of the patient.

2. The ventilation monitoring system of claim 1, comprising the sensor, wherein the sensor is positioned along a tracheal tube.

3. The ventilation monitoring system of claim 2, wherein the sensor is positioned along the tracheal tube at a location that is configured to be inserted into a body passage of the patient during the mechanical ventilation procedure.

4. The ventilation monitoring system of claim 2, wherein at least a portion of the sensor is exposed on an exterior surface of the tracheal tube.

5. The ventilation monitoring system of claim 2, wherein a body of the sensor is molded into the tracheal tube.

6. The ventilation monitoring system of claim 1, comprising the sensor, wherein the sensor is coupled to a mask that is configured to non-invasively direct respiratory gases from a ventilator to the patient.

7. The ventilation monitoring system of claim 6, wherein at least a portion of the sensor is exposed on an interior surface of the mask.

8. The ventilation monitoring system of claim 7, wherein the sensor is positioned at a bottom portion of the mask.

9. The ventilation monitoring system of claim 1, wherein the one or more processors are configured to determine a baseline ratio for the patient during an initial portion of the mechanical ventilation procedure and to calculate the expected ratio based on the baseline ratio.

10. The ventilation monitoring system of claim 1, wherein the one or more processors are configured to monitor a trend in the detected ratio over time and to predict a future condition of the patient based on the trend.

11. The ventilation monitoring system of claim 10, wherein the one or more processors are configured to use machine learning to predict the future condition of the patient based on the trend.

12. The ventilation monitoring system of claim 1, comprising a ventilator with a display screen, wherein the one or more processors are configured to output the notification and ventilation parameters for display on the display screen.

13. A method of operating a ventilation monitoring system, the method comprising:
receiving, at one or more processors, data from a sensor positioned along a breathing circuit during a mechanical ventilation procedure for a patient;
analyzing, with the one or more processors, the data to determine a detected ratio of respective concentrations of ions at the sensor;
comparing, with the one or more processors, the detected ratio to an expected ratio; and
outputting a notification in response to determining that the detected ratio does not correspond to the expected ratio.

14. The method of claim 13, comprising:
determining, with the one or more processors, a baseline ratio for the patient during an initial portion of the mechanical ventilation procedure; and
calculating, with the one or more processors, the expected ratio based on the baseline ratio.

15. The method of claim 13, comprising:
receiving, at the one or more processors, patient data comprising physiological parameters, gender, age, smoking history, or any combination thereof; and
calculating, with the one or more processors, the expected ratio based on the patient data.

16. The method of claim 13, comprising:
monitoring, with the one or more processors, a trend in the detected ratio over time; and
predicting, with the one or more processors, a future condition of the patient based on the trend.

17. A ventilation monitoring system, comprising:
a tracheal tube comprising a sensor configured to generate data indicative of respective concentrations of ions;
a ventilator configured to couple to the tracheal tube and comprising one or more processors configured to:
instruct delivery of respiratory gases through the tracheal tube during a mechanical ventilation procedure for a patient;
receive the data from the sensor during the mechanical ventilation procedure;
analyze the data to calculate a detected ratio of the respective concentrations of the ions;
compare the detected ratio to an expected ratio to determine a condition of the patient; and
output a notification via a display screen of the ventilator to indicate the condition of the patient.

18. The ventilation monitoring system of claim 17, wherein the sensor is positioned along the tracheal tube at a location that is configured to be inserted into a body passage of the patient during the mechanical ventilation procedure.

19. The ventilation monitoring system of claim 17, wherein the one or more processors are configured to monitor a trend in the detected ratio over time and to predict a future condition of the patient based on the trend.

20. The ventilation monitoring system of claim 17, wherein the ions comprise sodium ions and potassium ions, and the detected ratio and the expected ratio relate the respective concentrations of the sodium ions and the potassium ions to one another.
